(19) United States
(12) Patent Application Publication
(10) Pub. No.: US 2010/0249540 A1
(43) Pub. Date: Sep. 30, 2010

(54) Medical Monitoring System With Open Device Architecture

(75) Inventor: Daniel Lisogurski, Boulder, CO (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/725,957

(22) Filed: Mar. 17, 2010

Related U.S. Application Data
(60) Provisional application No. 61/165,254, filed on Mar. 31, 2009.

(51) Int. Cl. A61B 5/00

(52) U.S. Cl. 600/301; 600/300

(57) ABSTRACT

According to embodiments, systems and methods for monitoring multiple physiological parameters made available by multiple sensors positioned on a patient are provided. The system may include a plurality of sensors, a multi-parameter monitor and a plurality of displays. The monitor may include a plurality of sensor interfaces, a multi-parameter processor and an output interface. The sensors connect to the sensor interfaces and generate physiological signals that are transmitted to the processor for processing. The processed signals are transmitted to at least one display for viewing by medical personnel. The sensor interfaces are of like kind and provide easy connection and disconnection for exchange of sensor types, such as ECG, oximetry, body temperature and NIBP. The monitor may communicate with the plurality of displays. When a patient is transported from one location to another, the monitor can be transported with the patient and operatively link to displays in the new location thus eliminating the need to transport multiple devices or machines from one location to another.

### RELATED APPLICATION

This application claims the benefit of U.S. Provisional Application No. 61/165,254 filed Mar. 31, 2009, which application is hereby incorporated herein by reference.

### BACKGROUND

The present disclosure relates generally to medical monitoring devices, and more particularly to apparatus, methods and systems for monitoring and displaying multiple physiological parameters presented from a patient.

In modern medicine and modern healthcare facilities, the presence of and need for specialized patient care monitors continues to increase. These monitors continue to be state of the art, microprocessor driven devices critical to the precise care of the patient. It is often the case that modern physiological sensors and monitors present data in a variety of ways that can impart critical information regarding a patient's condition. For example, sensors and monitors can relate critical data related to pulse oximetry, airway management, patient temperature, heart rate, and blood oxygen levels as well as others.

Often, more than one of these devices monitors a patient's condition at a particular point in time. The need for physicians and medical personnel to have access to these physiological parameters can be life-critical. Medical personnel can understand and monitor a patient's physiological well-being based on this information, which can assist in determining an appropriate course of action.

Typically, current modern sensors and monitoring systems and apparatuses are designed with a proprietary connector for each type of sensor. Each connector between a sensor and a monitoring system links to a hard-wired circuitry or to a module which may control the sensor and extract useful information desired by medical personnel. The data and information from multiple sensors is commonly combined on a display for usage by the attending medical personnel. FIG. 1 shows a multi-parameter monitor which may be utilized in medical situations. A user interface and display 10 receives signals from a processor 20. The user interface and display publish the resulting information to medical personnel. The processor 20 receives information and data from a plurality of specialized sensor modules, which may be purchased from a third party. Alternatively, the processor 20 may directly interface with one or more sensors. In the example shown, the specialized processors are an ECG processor 30, an oximetry processor 31, a noninvasive blood pressure (NIBP) processor 32 and a temperature processor 33. The specialized processors in turn receive individual and specialized signals sensed by a plurality of specialized sensors. These specialized sensors including ECG sensor 40, oximetry sensor 41, blood pressure cuff 42 and temperature sensor 43 connect to the respective processor 30, 31, 32, 33 using proprietary connectors. The Figure shows a proprietary ECG connector 50, a proprietary oximetry connector 51, a proprietary NIBP connector 52 and a proprietary temperature connector 53.

As medical technology advances, more technology is utilized in a wider variety of manners. It is foreseeable that additional technologies may be introduced into a medical environment. It is foreseeable that current methods or utilities of measuring certain physiological parameters may not have been conceived when a healthcare facility purchased their existing monitors. It is also foreseeable that current physiological parameter measurements may be replaced by new physiological parameter measurements that are not yet contemplated but may enable better medical care decisions and outcomes.

As devices to measure additional parameters are added to operating rooms and other health care facilities, intensive care units and other medical providers must learn to operate and configure a multiplicity of devices. These additional devices lead to more cluttered work areas and tangles of patient cables. The increase in dedicated and proprietary interfaces will also force these units and providers to navigate an ever increasing range of user interfaces while manually consolidating and recording vital signs or silencing alarms which may be not useful or redundant due to an overall medical assessment.

The lack of communication between monitors may make it challenging to configure intelligent alarms. Due to the increasing and large number of false or redundant alarms, healthcare workers may disable or ignore events which could be critical to a patient's well being. A monitor connected to a plurality of sensors may better prioritize alarms. For example, a loss of pulse detection from a previously noisy oximetry sensor may be of less concern to a care provider if the patient's ECG and respiration are stable. However, a loss of respiration, QRS complexes, and oxygen desaturation within a short period of time may trigger a very high priority alarm.

Multiple parameter monitoring systems were designed in their current manner because it was not typically practical for a patient to wear a sensor which included all of the electronics to do its own processing. For instance, a patient may wear an ECG sensor containing electrodes connected to a cable, but any signal amplification, filtering, digitization or digital processing occurs within the monitor.

Accordingly, there may be a need for small stand-alone sensors which are wearable by the patient and may process their own data. These modern sensors will have the capability to communicate to one or more monitors through a standard wired or wireless interface.

### SUMMARY

In an embodiment of the disclosure, a monitoring system for physiological data may be provided. The physiological data is generated by sensors disposed on or in a patient. The embodiment also includes a multi-parameter monitor interfacing with the sensors. The monitor provides a connection point nearby a patient which can easily be transported with the patient. This eliminates the need to disconnect numerous cables or should the patient be transported from one area of a health care facility, such as an intensive care unit, to another area of the health care facility, such as an x-ray laboratory. The monitor communicates with a display and user interface to publish data provided by the sensors, processed by the monitor and used by medical personnel for decision making purposes.

In an embodiment, the sensors are connected to the monitor via a common connection means. By common, it is intended to indicate that the connectors are common between the sensors—that the sensors utilize a single or a minimum number of connection types. In this embodiment, the sensors all utilize the same connector type. For example, the sensors all connect to the monitor utilizing a Universal Serial Bus (USB) connection. It is recognized that there are many available options for suitable connection types. Some examples include Ethernet, Universal Asynchronous Receiver Transmitter (DART) or other proprietary means. Alternatively, the sensors are connected to the monitor via a common wireless link. For example, the sensors would all connect to the monitor utilizing IEEE 802.11 wireless networks. It is recognized that there are many options for a suitable wireless link. Some examples include Bluetooth, IEEE 802.15.4 and other proprietary means. The use of a common connection type promotes ease of exchange or addition of sensors without hardware modifications.

In an embodiment, the monitor is linked to one or more displays via a wired link, The displays receive measurements from the monitor for display. The displays are also capable of sending information back to the monitor for change of settings, such as to change the update rate of a non-invasive blood pressure measurement or altering the display parameters based on user preferences, In one embodiment, the display is easily transportable with the monitor when it is necessary to transport the patient to a different portion of the healthcare facility. It is alternatively possible that the display is easily disconnected from the monitor during transportation of the patient and the monitor can easily connect to a display located at the destination location in the health care facility.

Alternatively, the monitor is connected to the displays via a wireless link. The monitor is configured to provide a wireless signal that is detectable by the display in a particular location of the health care facility. For instance, when a patient enters a particular location in a health care facility, a display in that location will detect the proximity of the monitor traveling with the patient and automatically link to that monitor and display the physiological parameters associated with that patient. An authentication method may be used to prevent the unwanted display of information from another nearby patient, or to ensure that only authorized personnel are able to view said parameters.

As time goes by, current techniques and sensors may be replaced or augmented by new sensors that provide similar data, or which may provide monitoring of new physiological parameters. In one embodiment, a monitor may download new software and drivers for connecting with the new or augmented sensors. In this embodiment, each new sensor provides the necessary software and drivers. Memory, such as Flash or ROM, residing on the sensor itself may be read by the monitor. When a sensor is connected to a monitor, the monitor will detect a new device. The monitor will then automatically search the new sensor, or other areas, such as the Internet, for software and driver information and download the necessary information to enable communication between the two devices and correct processing and display of parameters. However, for cost sensitive or disposable devices, this may increase costs.

Alternatively, the monitor and sensor will operate under a cooperative protocol by which the sensor will exchange configuration information with the monitor. Such a protocol may be an open standard allowing any sensor manufacturer to interface with the monitor. Alternatively, the protocol may be proprietary to prevent untested or unqualified sensors from interacting with the monitor and displays. Such a protocol may include the patient Identification information associated with the sensor, the number of channels supported by the sensor, and for each channel the available information may include the class of data from a known set if applicable (e.g. heart rate, ECG, cardiac output, blood pressure) or “new sensor type”, the type of display required for each channel (e.g. precision, update rate, title, and unit requirements for measurements such as heart rate or temperature), sensor alarm status information (e.g. default alarm limits and severity). The monitor may configure alarms such as heart rate from all sources with the same range, and alarm intelligently based on known sensor types. It may also compare heart rate from different sources.

In order to maintain simplification of sensor devices, the software and drivers may be downloadable by the staff at a health care facility. Upon receipt of a new or augmented sensor, the health care facility will also receive a means for downloading the appropriate software and drivers to its monitors. It is understood that there are numerous to upgrade the software, including at least memory storage devices and wired or wireless networks. Once the health care facility has obtained the appropriate software and drivers, it can download it to all of its monitors so that they are ready to connect and communicate with the new or augmented sensors. It is understood that this may be an automated process which upgrades a large number of monitors at once via a network connection such as Ethernet, or a mesh network such as IEEE 802.15.4.

Within any medical environment, there may be multiple health care professionals that need to access and view physiological data simultaneously in relation to one patient. An example of this would be in an operating room where an anesthesiologist needs to access a certain set of parameters and data, the operating physician needs to access a different set of parameters and data and a third professional needs to access a third set of parameters and data. Each professional has their own preferences and needs. As such, in one embodiment, each professional would be able to customize a display for their use. In the operating room example, multiple displays would provide individualized viewing for critical health care providers. The anesthesiologists could customize their display as well as could the surgeon and other health care providers.

The customization of viewing discussed above could be automated in one embodiment. For instance, the anesthesiologist would be able to determine his or her preferences in advance and save those preferences. The saved preferences would be downloadable to the monitors. Upon entering the operating room the anesthesiologist would enter a personal identification into the system that would recognize the anesthesiologist and provide the appropriate preferences to the display. Alternatively, a medical provider would carry a personal identification device identifying the operator, much like a typical identification badge carried by a vast number of persons employed by any number of corporations in the world. Upon entering within a proximity of the patient's monitor, the monitor would sense the presence of the medical provider and select that provider's preferences for display. A priority system could select the appropriate preferences in the instance when multiple providers were within proximity to the monitor.

A single monitor system will be capable of consolidating, time synchronizing, and logging all data from the patient for display. In one embodiment, a display is mounted in the patient's room. Alternately, a display is attached to the patient's bed. When attached to the bed, it easily travels with the patient to provide constant communication with the monitor and display constant critical information during transport.

When a patient is moved from one portion of the medical facility to another, no burdensome disconnection and reconnection of sensor wires is required. The portable display would suffice for transport until a larger display mounted in the destination location connects to the monitor and displays the necessary physiological data. This automatic transfer of connections provides invaluable time savings in a medical facility where time is of the essence.

Many other objects, features and advantages of the present disclosure will be evident to those of ordinary skill in the art from the following more detailed description, particularly when considered in light of the accompanying drawings and the appended claims.

### BRIEF DESCRIPTION OF THE DRAWINGS

Illustrative embodiments of the disclosure are illustrated in the drawings, in which:

FIG. 1 illustrates a multi-parameter sensor system;

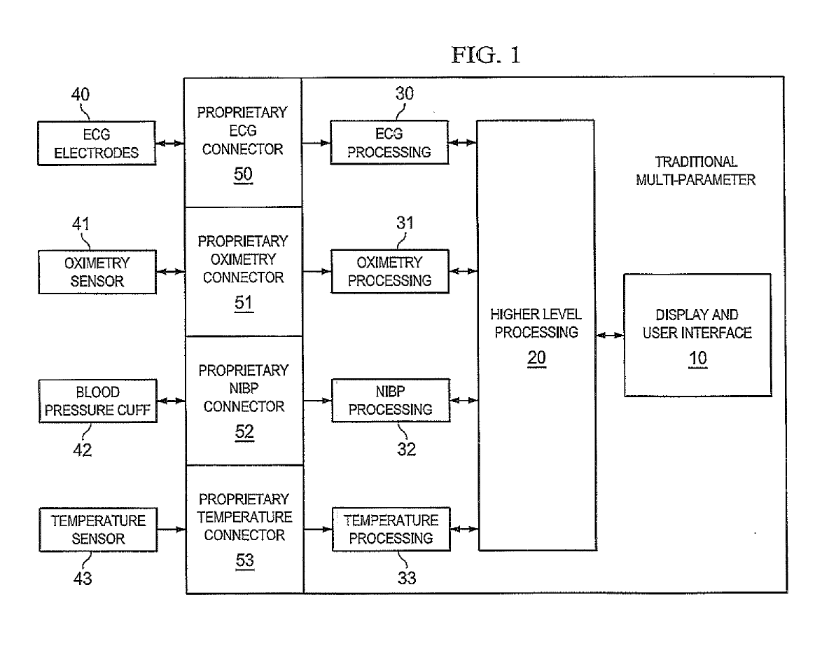

FIG. 2 illustrates an embodiment of a universal multi-parameter monitor and display system;

FIG. 3 illustrates an embodiment of an alternative universal multi-parameter monitor and display system; and

FIG. 4 illustrates an embodiment of a health care facility setup for a monitor and display system.

### DETAILED DESCRIPTION

In one embodiment, the present disclosure provides a system and method for a universal multi-parameter monitor providing increased flexibility to health-care providers as well as simplicity of use with regard to communication between a plurality of sensors and a monitoring system. A plurality of sensors will communicate with a monitor system in a common manner and allow increased mobility and constant monitoring of patients during transport to different parts of a facility. A monitoring system will communicate with and provide information for display to a controlled set of visual displays. The display of viewable information can be customized so that it provides information specifically relevant or preferential to a particular health-care provider or to a category of health-care providers such as a nurse or anesthesiologist or cardiologist.

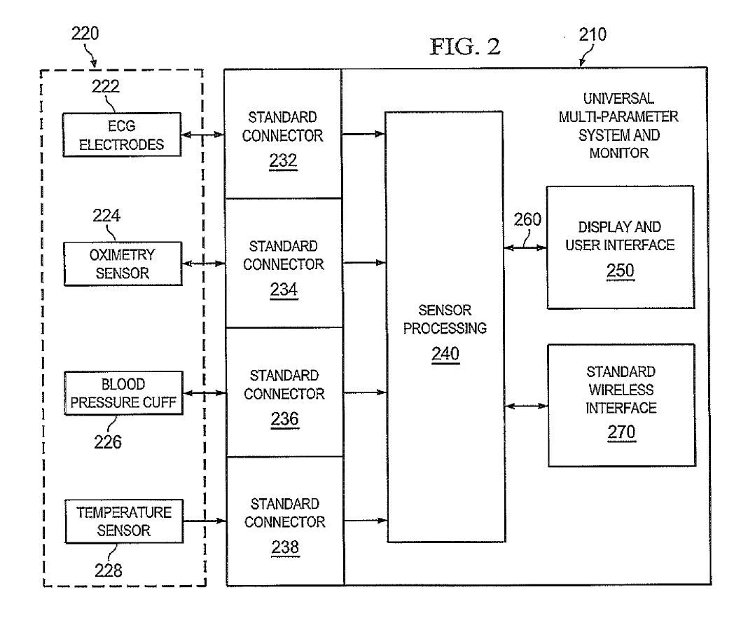

Referring to FIG. 2, an embodiment of a physiological parameter sensing and monitoring system 210 is shown. The system 210 may include a plurality of sensors 220 connected to a patient (not shown). ECG electrodes 222 attached to connector 232 provide an electrocardiogram. An oximetry sensor 224 attached to connector 234 provides pulse rate and oxygen saturation. A Non-Invasive Blood Pressure (NIBP) cuff 226 attached to connector 236 provides blood pressure information. A body temperature monitor 228 attached to connector 238 provides temperature information. Four connectors are shown in the example in FIG. 2, however it is understood that fewer or more sensors and connectors could be provided, and that different sensor types can be connected to a patient to provide information to a health-care provider. The sensors 220 interface with a processor 240, via the connectors 232, 234, 236 and 238. The monitor 240 processes the signals provided by the sensors 220. The connectors 232, 234, 236, 238 provide the connection between the sensors 220 and the processing unit or monitor 240. In this embodiment, the connectors, or interface means, 232, 234, 236, 238 are integral to the monitor 240 such that they are provided in the same over all “box” or unit. Alternatively, the connectors, or interface means, 232, 234, 236, 238 may be physically separated from the monitor 240 so that it comprises a separate unit or hub communicating with the processor.

A hub, such as a USB hub, is a peripheral that allows many devices to be connected to a single USB port on the host or monitor or on another hub. Hubs are often built into the host or monitor. However, it is possible to separate the hub from the monitor or host and provide a separate connection between the hub and monitor or host.

According to an embodiment, the connectors 232, 234, 236 and 238 between the sensors 220 and the monitor 240 may be all common to each other. For example, the connectors 232, 234, 236 and 238 operate in accordance with the USB standard. USB is a serial bus standard to connect devices to a host computer. USB allows many peripherals to be connected using a single standardized interface socket and to improve plug and play capabilities by allowing hot swapping; that is, by allowing devices to be connected and disconnected without rebooting a processing unit or turning off the device. Other convenient features include providing power to low-consumption devices, eliminating the need for an external power supply; and allowing many devices to be used without requiring manufacturer-specific device drivers to be installed. USB is intended to replace many varieties of serial and parallel ports that otherwise may exist in a multi-parameter monitoring system. One skilled in the art will realize that there are suitable alternatives to USB for a wired interface. For example, Ethernet and a Universal Asynchronous Receiver Transmitter (UART) are examples of available connection types. It is also understood that in addition to the requirements for USB, Ethernet or other standards, electrical isolation may be required in the monitor and sensor to ensure the safety of the patient.

Alternatively, it is envisioned that a limited number of interface types for the connectors 232, 234, 236, 238 can be utilized. Although a certain amount of simplicity in using a single interface type for the connectors 232, 234, 236, 238 may be sacrificed, flexibility is gained by allowing more than one type of interface. It is recognized that the number of types of interfaces should be capped at an acceptable level. Any number more than two types of interface may defeat the simplicity obtained through the disclosure. However, it is understood that the number of sensor interface types is not otherwise limited.

In an embodiment, the processor 240 conditions the received signals in accordance with appropriate standards and prepares the resulting data for transmission to a display and user interface 250. This processing of signals received from sensors via wired connectors 232, 234, 236, 238 and may include signal amplification, filtering, digitization and digital processing. Alternatively, where it is practical to embed electronics and processing devices with the sensors 220, wherein the sensors process, at least partially, their own data.

The transmission from the processor 240 to the display and user interface is via a wired connection 260. Similar to the interfaces between the sensors 220 and the monitor 240, the interface 260 between the monitor 240 and the display and user interface 250 is wired in this embodiment of the disclosure. The display interface 260 is similarly one of any common type in use today and is understood by those skilled in the art. Also, the monitor 240 is capable of interfacing with a plurality of display and user interfaces 250. The interface 260 between the monitor 240 and the display and user interfaces 250 is a two way communication interface which enables the user to provide inputs which are transmitted to the monitor 240. Acceptable communication interfaces include those previously discussed, such as an USB, or a FireWire type interface. Examples of inputs which a user, via the user interface 250, would provide include display parameters to meet medical standards, the preferences of a particular user and medical provider identification.

Still referring to FIG. 2, sensors 220 may be disconnected from monitor 210 without adverse effect on any other sensor processing that may be undergoing in the monitor 210. This is due to the hot swapping characteristics of many interface types, such as USB. Also, additional sensors can be connected to the monitor 210 during processing of the data from other sensors 220. Due to the universal aspect of the monitor 210 and the standardized connectors 232, 234, 236, 238 utilized to connect the sensors 220 to the monitor 210, the monitor 210 will detect the attempt to interface a new sensor. Each sensor 220 will possess an identification mechanism that is common with the type of sensor interface being utilized. The processor 240 recognizes the type of sensor being connected and connects accordingly. The processor 240 processes the data received from new sensor 220 utilizing any necessary software that was downloaded to the monitor 240 either via a “plug-in” module or remotely. This is discussed more fully later in this description.

A standard wireless interface 270 connects to an external source capable of downloading software to processor 240. The wireless interface 270 may connect to a central control (not shown) located at a remote location. Possible remote locations include an administrative post within the health-care facility such as a nursing station or external to the healthcare facility and responsible for maintaining current software on monitors 210 under its control. Upon receipt by a health care facility of a new or augmented sensor, the central control station downloads software to all relevant monitors in the facility via the wireless interface.

Alternatively, any software required for proper interfacing of the sensors 220 resides on each new sensor that may be connected to the monitor 210. The sensor 220 stores a “plug in” software module on itself, which is capable of downloading and installing by the processor 240. The sensor 220 exchanges configuration information with the processor 240. This exchange operates in line with a protocol which may be an open standard allowing any sensor manufacturer to interface with the monitor 210. Alternatively, a proprietary protocol prevents untested or unqualified sensors from interacting with the monitor 210.

As is known in the art, a standard connection type such as USB is linked through a series of hubs. When a USB device is first connected to a USB host or monitor, the USB device enumeration process is started. The enumeration starts by sending a reset signal to the USB device. The speed of the USB device is determined during the reset signaling. After reset, the host reads configuration information from each USB device, and the device is assigned a unique address. If the device is supported by the host, the devices' drivers needed for communicating with the device are loaded and the device is set to a configured state.

In another embodiment, a plurality of sensors will communicate with a monitor system in a common manner and allow increased mobility and constant monitoring of patients during transition to different parts of a facility. A monitoring system will communicate with a plurality of sensors and provide information for display to a controlled set of visual displays. The display of viewable information is configured to provide relevant information to the appropriate health-care provider.

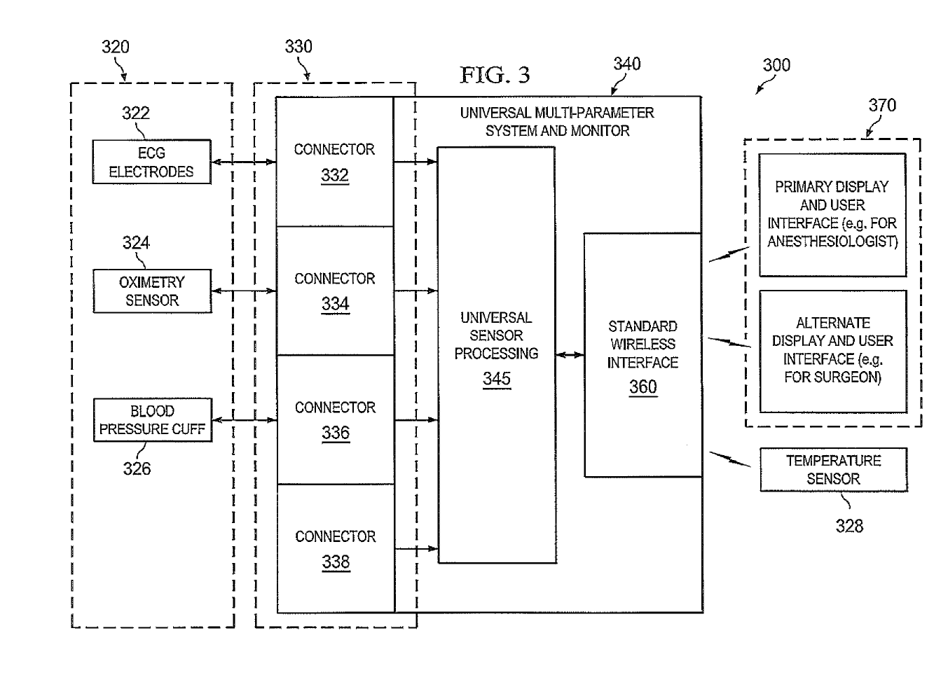

Referring to FIG. 3, an embodiment of a physiological parameter sensing and monitoring system 300 is shown. The system 300 includes a plurality of sensors 320, a monitor 340 and a plurality of display and user interfaces 370. The sensors 320 are connected to a patient (not shown). The sensors 320 connect to the monitor 340 by means of the connectors 330. ECG electrodes 322 provide cardiac information. An oximetry sensor 324 provides blood chemistry information. A NIBP sensor 326 provides blood pressure information. System 300 also comprises a body temperature monitor 328 which provides body temperature information. Three sensors are shown in the example in FIG. 3 as part of the group of sensors 320, however it is understood that fewer or more sensors can be connected to a patient to provide critical health information to a health-care provider. Also shown in FIG. 3 is a connector 338. This connector 338 is connectable to a new or augmented sensor. It is understood that FIG. 3 illustrates an embodiment where the connectors 330 can be either a wired or a wireless set of connectors—or a combination of wired and wireless. Alternatively, where a plurality of wireless sensors is used, it is understood that the wireless sensors 320 would connect to a standard wireless interface 360. This wireless interface 360 provides connectivity to all of the wireless sensors 320 as well as to the displays 370 as more fully described below.

In an embodiment, the monitor 340 comprises a plurality of connectors 330. In this example, there is an ECG connector 332, an oximetry connector 334, an NIBP connector 336 and a body temperature connector 338. It is understood that each interface 330 is not dedicated in that it is capable of disconnecting from the sensor 320 shown and connecting to a new or different sensor 320. The monitor 342 further comprises a processor 345 connected to the connectors 330. The processor 345 connects to a wireless interface 360. The wireless interface 360 connects wirelessly to a plurality of display and user interfaces 370. It is understood that more or less than the two displays and user interfaces shown are connectable to the monitor 340 via the wireless interface 360.

The sensors 320 interface with the universal multi-parameter monitor 340 via the connectors 330. The connectors 330 between the sensors and the monitor 340 all support a common interface type. For example, the interfaces 330 all operate using a USB connection. One skilled in the art will realize that there are again suitable alternatives to USB for an interface. For example, Ethernet and Universal Asynchronous Receiver Transmitter (UART) are examples of available connection types. As described above, it is also understood that wireless links may replace, or be used in parallel with, the standard connectors 330. Suitable wireless interface technologies include IEEE 802.11 wireless networks, Bluetooth, IEEE 802.15.4 as well as other proprietary alternatives.

As shown in FIG. 3, multiple sensors 320, 328 interface with the connectors 330 and with the monitor 340. It is an aspect of the current disclosure that sensors may interface with the monitor 340 via either the wired connectors 330 or the wireless interface 360 or both. Alternatively, some, or all, of the connectors 330 are wireless interfaces and the sensors 220 interface with the connectors wirelessly. Such interfacing means by the sensors will further eliminate cable clutter and confusion in the medical environment, Accordingly, as an example, sensor 328 is shown interfacing with the monitor 340 via the wireless interface 360. It is understood by those skilled in the art that more, fewer, or all, of the sensors 320 may interface wirelessly with the monitor 340 via the wireless interface.

It is understood that whereas some sensors may logically use wireless sensor interfaces, other sensor types may logically necessarily continue to utilize wired interfaces, For example an SPO2 sensor may be a small wireless device, but defibrillation electrodes which can both monitor or shock a patient need to generate 10's of amps of current at thousands of Volts. Currently defibrillation electrodes more likely utilize a wired interface due power consumption rationales. Accordingly, in one embodiment of the disclosure, the monitor has means for wireless and wired connectivity to accommodate a plurality of sensors using either or both methodologies.

The monitor 340 provides a connection point nearby the patient (not shown) which can easily be transported with the patient. Accordingly, the monitor 340 can attach to the patient or alternatively it can attach to the bed or transportation device (not shown) used by a health care facility. Transportation of the monitor 340 along with a patient eliminates the need to disconnect cables or re-located displays if, for instance, the patient is transported from one portion of a health care facility to another.

In an embodiment, the universal sensor processor 345 connects to an interface means 360. In this instance, the interface means 360 is a wireless interface. One skilled in the art will understand that there are a multitude of suitable options for a wireless interface 160. For example, suitable wireless interface technologies include IEEE 802.11 wireless networks, Bluetooth, IEEE 802.15.4 as well as other proprietary alternatives.

The interface means 360 provides a connection between the universal sensor processor 345 and a primary, or secondary, display 370. The display 370 show the processed information gathered by the sensors 320 and processed by the universal sensor processor 345.

In the embodiment in FIG. 3, the universal multi-parameter monitor 340 is a portable device that is easily transported with a patient from one portion of a facility to another. The wireless interface 360 allows the monitor 340 to easily connect to a display 370 that is within proximity to the monitor 340. FIG. 3 shows a second, alternative display 370 connected to the monitor 340 via the wireless interface 360. It is possible that more displays are connectable to the monitor 340. For instance, it would be convenient for multiple displays to be located in an operating room. An anesthesiologist is likely interested in a different set of data from a chief surgeon or from another medical provider within an operating room. Multiple displays allow each healthcare provider to view their sensor data preferences individually without interfering with the other healthcare providers. Each display is configurable to display the sensor data of interest and more than one control panel may be available to configure the displays or silence alarms without walking around the patient's bed.

Accordingly, each display 370 may be capable of communicating with the monitor 340 via the wireless interface 360. A user is capable of providing inputs to the primary display 370 for, for instance, to indicate a preferred setting. Moreover, the displays 370 are capable of providing a signal that is sensed by the monitor 340. Each display 370 may provide a signal that identifies the display 370 and provides initial preference settings to the monitor 340.

Alternatively, each area or room of the healthcare facility may provide a wireless signal detectable by a monitor 340 that allows a monitor 340 to identify what location or room it is in and provide the appropriate preferences for that location. For instance, if the monitor 340 is moved along with a patient into an operating room, the monitor 340 may detect a wireless signal from the operating room that allows it to determine that it has entered predetermined limited range of the operating room locale and provide the appropriate preferences to the displays 370 in the room. Each display 370 will have been preset to indicate the appropriate preferences.

It is understood in the art how two different devices will recognize each other over a wireless network. A Wireless Local Area Network (WLAN) is a wireless alternative to a computer LAN that uses radio instead of wires to transmit data back and forth between two devices, or multiple devices. Wi-Fi is a commonly used wireless network in computer systems to enable connection to devices that have Wi-Fi functionalities. Wi-Fi networks broadcast radio waves that can be picked up by Wi-Fi receivers attached to different computers or mobile phones. Fixed wireless Data implements point to point links between computers or networks at two locations, often using dedicated points. These are just some examples of wireless networks and it is understood that any wireless network is acceptable if it provides suitable confidentiality, data protection and reliability.

Additional to the portable aspects of the disclosure described above and to the ease of connection to a display system in a defined location such as an operating room or a recovery room, the monitor 340 also communicates with a central system (not shown). This allows the sensors 330 to be monitored from a remote location, such as a nursing station or a more senior physician keeping track of the status of multiple patients.

In another embodiment, the primary display 370, or one of the secondary displays 370, is also portable and attachable to the bed or transportation vehicle of the patient (not shown) such as a wheel chair. The single monitor 340 associated with a patient would consolidate, time synchronize and log all data from the patient. When the patient is moved from one location, such as an operating room, to another location, such as ICU, no sensors 320 need to be disconnected and the portable bedside display 370 provides constant information during transport or until a larger display 370, which is more permanently mounted in a destination area, can communicate with the processor 340 via the wireless interface 360.

In the embodiment shown in FIG. 3, the standard wireless interface 370 is connectable to an external source capable of downloading software to the monitor 340. Much like the embodiment described in relation to FIG. 2, downloaded software will be for purposes of operating any new sensor 320 or updated sensor 320. In the embodiment shown in FIG. 3, the wireless interface 360 connects to a central control station (not shown) located at a remote location. The remote location could be an administrative post within the health-care facility or external to the healthcare facility and responsible for maintaining current software on monitors 340 under its control. Upon receipt by a healthcare facility of a new or augmented sensor, the central control station downloads software to all relevant monitors in the facility via the wireless interface.

Alternatively, the software necessary to download required for proper interfacing of the sensors 320 resides on each new sensor that may be connected to the monitor 340. In this embodiment, the sensor 320 stores a “plug in” software module on itself, which is capable of downloading and installing by the monitor 340. The sensor 320 exchanges configuration information with the monitor 140. This exchange operates in line with a protocol which may be an open standard allowing any sensor manufacturer to interface with the monitor 340. Alternatively, a proprietary protocol is used to prevent untested or unqualified sensors from interacting with the monitor 340 and display 370.

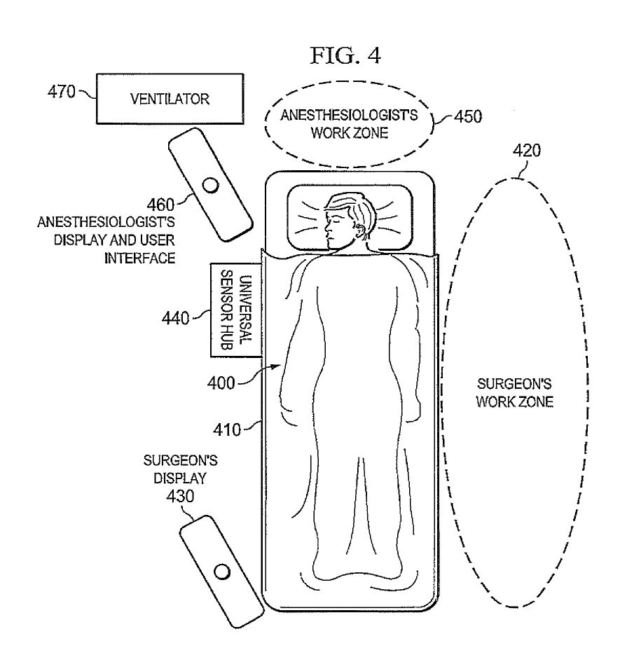

FIG. 4 shows an example operating room facility using an embodiment of the disclosure described in relation to FIG. 3. A patient 400 may be located in the center of the room on an operating table 410, A surgeon's work zone 420 is next to and abutting the operating table 410 and the patient 400. A surgeon's display and user interface 430 is located in a convenient location relative to the surgeon's zone 420. A universal sensor monitor 440 is located on the side of the table 410 opposite the surgeon's zone 420. An anesthesiologist's zone 450 is located at one end of the table 410 and an anesthesiologist's display and user interface 460 is located in a convenient location appropriate for viewing by an anesthesiologist.

FIG. 4 also shows a ventilator 470. In an embodiment, dedicated hardware such as a ventilator 470 or an infusion pump (not shown) can communicate with the monitor 440. The monitor 440 will display settings and status or be controlled from to one of the display and user interfaces 430, 460 or from a central location (not shown). The ventilator 470 may contain a wireless interface (not shown) that allows it to communicate with the monitor 440.

It is understood that the zones and locations described in regard to FIG. 4 are examples and one skilled in the art, for example a surgeon, will have preferences for display and monitor locations.

Upon completion of the surgery, the patient 400 will be moved to a new location in the healthcare facility. The multiple displays 430, 460 do not need to be disconnected from the patient 400 or from the table 410. The patient 400 can be easily moved to a new location where display and user interfaces will connect when the patient 400 is in the new location. During transport of the patient 400, a portable display and user interface (not shown) can be attached to the table 410 to allow constant monitoring. Automatic recognition of a monitor that comes within a proximity to a display and user interface will cause the monitor 440 and display to connect via a wireless interface.

While various embodiments have been described in this application for illustrative purposes, the claims are not limited to the embodiments described herein. Equivalent devices or steps which operate according to principles of the present disclosure may be substituted for these described, and thus fall within the scope of the claims that follow:

What is claimed is:

1. A system for monitoring physiological data of a patient, the system comprising:
a monitor, said monitor comprising at least one processor interface and a sensor processor for receiving and processing data from said processor interface; and
at least one sensor, said sensor being operable to sense a parameter of interest related to the patient;
wherein said at least one sensor operatively links to said monitor by said processor interface, whereby data is transmitted from said sensor to said sensor processor; said data is processed by said processor and the processed data is transmitted to an at least one display and user interface by the processor interface.

2. The system of claim 1, wherein said at least one of said at least one processor interface is operable to link to at least one wireless sensor.

3. The system of claim 2, wherein said at least one display and user interface is a wireless device and said at least one wireless processor interface is operable to link to said display and user interface.

4. The system of claim 3, wherein said display and user interface comprises a screen and a user input mechanism, said user input mechanism being functional for sending user inputs to said sensor processor.

5. The system of claim 4, wherein the user inputs include at least viewing preferences, display identification data and parameter selection information relating to sensor data processed by said monitor.

6. The system of claim 5, wherein the monitor provides outputs via the processor interface to at least one of the at least one display.

7. The system of claim 6, wherein at least one of the at least one display and user interface sends a signal receivable by a monitor within a predetermined proximity, said signal indentifying the at least one display and user interface with an identification parameter and providing initial setting preferences to the monitor.

8. The system of claim 7, wherein the at least one display disconnects from the monitor when the monitor exceeds the predetermined proximity in relation to the at least one display and user interface.

9. An apparatus for monitoring physiological data generated by an at least one sensor located on or in a patient, the apparatus comprising:
at least one processor interface, said at least one processor interface being operatively connectable to said at least one sensor; and
a sensor processor, said processor being able to process data received from the at least one sensor via the at least one processor interface;
wherein said apparatus is portable and connects via the at least one interface to an at least one display and user interface.

10. The apparatus of claim 9, wherein said at least one of said at least one processor interface is operable to link to at least one wireless sensor.

11. The apparatus of claim 10, wherein said at least one wireless processor is operable to link to at least one wireless display and user interface.

12. The apparatus of claim 11, wherein said apparatus further comprises at least one display and user interface.

13. The apparatus of claim 12, wherein said apparatus can be attached to a transportation device.

14. The apparatus of claim 13, wherein said monitor connects in a generally continuous manner to a portable display during transport from one location of a healthcare facility to another location

15. The apparatus of claim 9, wherein the sensor processor is downloadable with software and drivers, said software and drivers providing connectivity with said at least one sensor, wherein the software and drivers are downloadable to said sensor processor via a plug-in module which resides on a sensor.

16. A method of monitoring multiple physiological parameters of a patient from signals generated from a plurality of sensors disposed on or in the patient, said method comprising;
connecting said sensors to a processor interface;
processing the signals received via the interface with a multi-parameter processor to obtain processed physiological data; and
transmitting the processed physiological data via an output interface to at least one display and user interface;
wherein the display and user interface will provide a combined viewer display of a set of the processed physiological data according to a predetermined preference.

17. The method of claim 16, further comprising the step of transmitting the processed physiological data via an output interface to a secondary plurality of display and user interfaces.

18. The method of claim 16, wherein said processor interface is operable to connect to wired and wireless devices.

19. The method of claim 18, further comprising the step of transmitting of the processed physiological data to a particular display and user interface ceases and transmitting of the processed physiological data to a different display and user interface initiates when the patient is transported from one location of a healthcare facility to another.
